(19) United States
(12) Patent Application Publication
(10) Pub. No.: US 2003/0078514 A1
(43) Pub. Date: Apr. 24, 2003

(54) Relaxation device utilizing visual and audio stimulation

(76) Inventor: Tony Gougassian, Malden, MA (US)

Correspondence Address: LAMBERT & ASSOCIATES, P.L.L.C., 92 STATE STREET, BOSTON, MA, 02109-2004, US

(21) Appl. No.: 09/982,193

(22) Filed: Oct. 18, 2001

(51) Int. Cl. A61B 13/00

(52) U.S. Cl. 600/558

(57) ABSTRACT

A portable relaxation device is provided which utilizes display of lights and sound effects to induce a relaxed state in the user. A head mounted device is worn on the head of the user and displays light signals on the display field in front of the eyes of the user. The earphones provide sound effects to accompany light display. The control unit is used to control intensity, frequency, and sequence of the light and sound. In a preferred embodiment the light signals are provided by a series of light source emitters arranged in a semi circle on display fields before each eye of the user. The light source emitters fade in and out in sequence creating an optical illusion of a point moving through space such as that of an oscillating pendulum. The resulting effect on the user is increased state of relaxation and improved perception of well being.

### FIELD OF THE INVENTION

This invention relates to relaxation devices and particularly to devices that use visual and audio stimulation to induce relaxed emotional state in the user.

In the past, people used various techniques and devices to bring about relaxation and improvement in the emotional state. The techniques vary greatly and they could range from physical exercise to meditation, aromatherapy to hypnosis. The goal of this invention was to provide a relatively small, portable, and easy to operate device that would bring about state of relaxation and improved perception of well being by the user. The device could also prove to be useful in management and treatment of a variety of psychological and neurological disorders such as depression and anxiety.

### SUMMARY OF THE INVENTION

The disclosed device serves the purpose of inducing a state of relaxation and well being in the user of the device. The two primary parts of the device are a head mounted unit and a control unit. The head mounted unit is worn on a head of the user in a manner akin to that of wearing glasses or virtual reality goggles. By placement of the head mounted unit on the head of the user, the user's filed of vision is blocked off from the ambient light. The head mounted unit has a display field located in front of the visual field of the eyes of the user. A variety of light signals are produced on a display field. The nature of the light signals as well as their intensity, frequency, and sequence are chosen for their ability to induce relaxation. The control unit allows for control of the type of light signals, as well as control over their intensity, frequency, and sequence. The light sequences presented on the display field are accompanied by sounds received by the user through earphones, which are integrated with the head mounted unit. The combined effect of the specifically chosen visual and audio signals could be highly effective in inducing relaxation and perception of well being.

### BRIEF DESCRIPTION OF DRAWINGS

These and other features, aspects and advantages of the present invention will become better understood with reference to the following description, appended claims, and accompanying drawings where:

FIG. 1 is a depiction of one embodiment of the device showing the head mounted unit connected to the control unit.

FIG. 2 is depiction of a preferred embodiments of the invention showing the head mounted unit with a plurality of light emitters positioned on the display field.

### DESCRIPTION OF THE INVENTION

This invention represents a relaxation device.

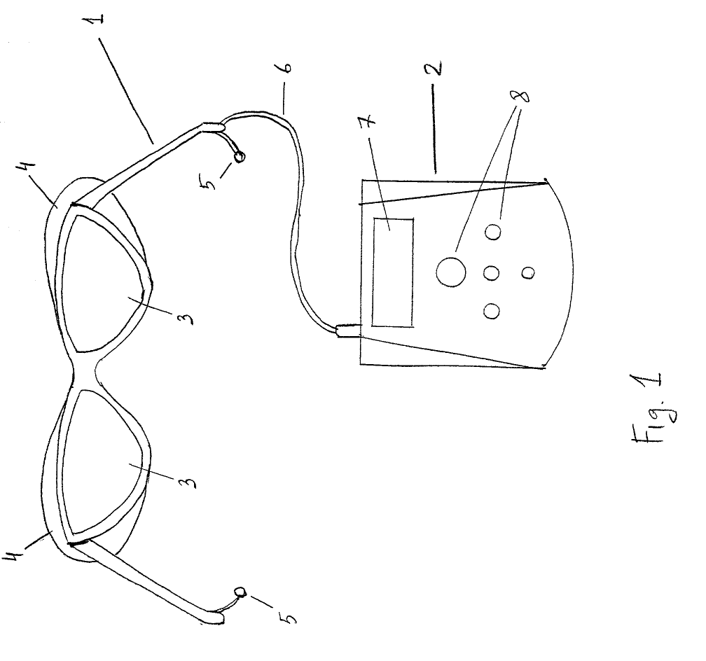

The basic components on the claimed relaxation device are presented in FIG. 1. The head mounted unit 1 could have a variety of shapes that are suitable for wearing over the eyes of the user. The skirt 4 could be used to serve the function of isolating user's field of vision from ambient light. This allows for better concentration by the user on the display filed 3.

The display field 3 is the area on the head mounted unit 1 which is directly in front of the user's field of vision. Light signals are displayed on the display field 3 via display means well known in the art. One potential embodiment could have a plurality of light sources emitters arranged on the display field 3. The light source emitters could be LEDs that are located throughout the display field 3. Such arrangement allows for almost infinite variety of light signal effects that could be produced on the display field 3 by the light source emitters acting in concert with each other.

The earphones 5 are integrated with the head mounted unit 1. The earphones 5 produce audio signals via audio means well known in the art.

The head mounted unit 1 is connected to the control unit 2 via cable 6 for transmission of control signals from the control unit 2 to the head mounted unit 1. The transmission of the control signals could be accomplished through any of the means well known in the art. For example, wireless means well known in the art could be used to transmit control signals from the control unit 2 to the head mounted unit 1. When wireless means are used, the control unit 2 has transmitting means well known in the art while the head mounted unit 1 has receiving means well known in the art for transmission of control signals from the control unit 2 to the head mounted unit 1.

The control unit 2 has actuators 8 used to control intensity, frequency, and sequence of the light display on the display field 3 and of the sound effects produced by the earphones 5. The design and method of operation of actuators 8 are well known in the art. The control unit 2 could have a display 7 for showing various information about the selected light and sound sequence.

During normal operation of the relaxation device, the head mounted unit 1 is worn on a head of the user in a manner that is akin to wearing glasses or virtual reality goggles. The user turns on and controls the device using actuators 8 on the control unit 2. The user is then presented with a potentially infinite number of light display sequences projected on the display field 3. The light displays are of a nature of production of a visual effect of moving light signals such as moving points, bands, or various other shapes. To compliment the light display, sound effects of various qualities are emitted through the earphones 5. The light and sound sequences are coordinated with each other and are chosen for their ability to induce relaxation and perception of well being in the user. Additionally, a human voice could be projected over the earphones, either alone by itself or in combination with other sound effects. The human voice is used to assist in attaining of the relaxed state by the user.

### DESCRIPTION OF THE PREFERRED EMBODIMENTS

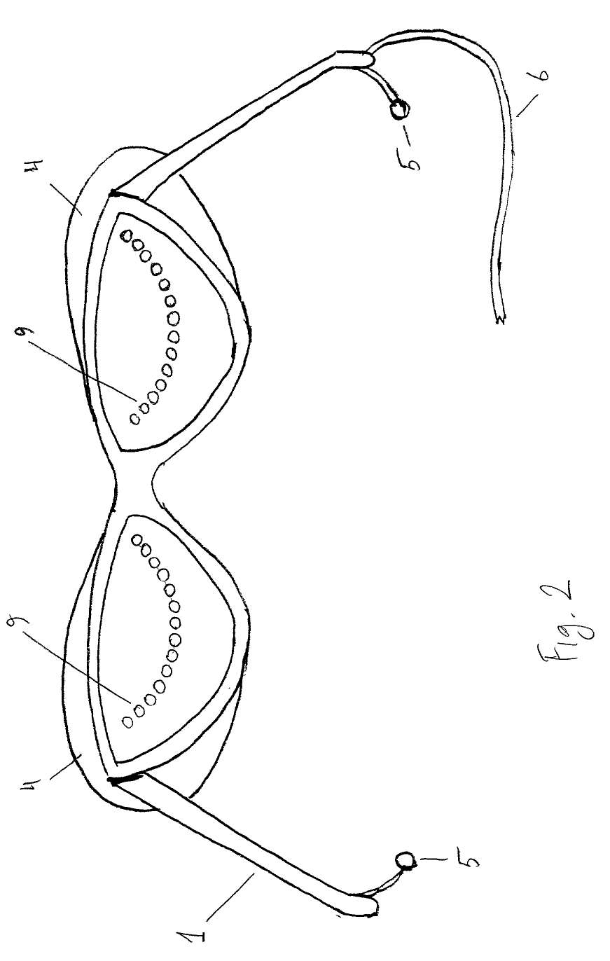

The preferred embodiment of the invention is shown in FIG. 2. A series of light source emitters 9 such as LEDs are arranged in a semi circular pattern on the display fields 3. As the light source emitters 9 oscillate in their intensity (fade in and out), they create an optical illusion of a moving point of light before each eye of the user. This point of light makes a motion similar to that of an oscillating pendulum. The light source emitters 9 are synchronized and produce the exact same display before each eye of the user. A sound effect such as sounds of an object moving through space may accompany the visual effect so as to further enforce perception of a pendulum moving through air. However, other types of sound effects could also be utilized. The control unit 2 is used to regulate properties of the illusory moving point of light, such as its intensity, speed of movement, and even color if such capability is desired.

The device works by putting a user in a state that is not unlike that of a hypnotic trance. As a result deep levels of relaxation could be achieved by the user. Alternating series of sequences could be preprogrammed wherein each sequence is characterized by specific color, intensity, and frequency. Subjecting the user to such alternating light and sound sequences enhances the relaxation effect on the user. Additionally, the relatively small size of the device allows it to be portable. It could be taken to work, on a business trip, or on vacation. The device might be of significant help to those who suffer from psychological and neurological ailments such as anxiety and depression.

What is claimed is:

1. Device for stimulation of visual and audio perception, comprising:
a head mounted unit;
a display field located on the head mounted unit and positioned before the field of vision of the user during operation of the device;
a display means integrated with the head mounted unit and producing light signals on the display field, wherein a visual effect created by the light signals is that of a motion by at least one light signal on the display field;
an audio means integrated with the head mounted unit and producing audio signals;
a control unit with actuators for regulating parameters and sequence of light signals produced by display means and audio signals produced by audio means, wherein control means are used to transmit control signals from the control unit to the head mounted unit.

2. Device of claim 1 wherein:
the head mounted unit has a shape that isolates the eyes of the user from ambient light.

3. Device of claim 1 wherein:
the head mounted unit has a skirt that isolates the eyes of the user from ambient light.

4. Device of claim 1 wherein:
the light signals are chosen for their ability to induce relaxation.

5. Device of claim 1 wherein:
the light signals are displayed on the display field in predetermined patterns.

6. Device of claim 5 wherein:
the predetermined patterns are chosen for their ability to induce relaxation.

7. Device of claim 1 wherein:
the display means has a plurality of light source emitters located on the display field.

8. Device of claim 7 wherein:
the integrated light source emitters are arranged in patterns on the display field wherein the integrated light source emitters emit light in specific predetermined sequences.

9. Device of claim 8 wherein:
the integrated light source emitters emit light that oscillates in a gradual manner.

10. Device of claim 8 wherein:
the predetermined sequences are chosen for their ability to induce relaxation.

11. Device of claim 7 wherein:
the integrated light source emitters are arranged in an identical single semi-circular pattern before each eye of a user wherein the integrated light source emitters emit light in specific predetermined sequences.

12. Device of claim 11 wherein:
the integrated light source emitters emit light that oscillates in a gradual manner.

13. Device of claim 11 wherein:
the predetermined sequences are chosen for their ability to induce relaxation.

14. Device of claim 11 wherein:
each of the semi-circular patterns produces a sequence of a synchronized and identical oscillating pendulum visual effect on the display field.

15. Device of claim 1 wherein:
the audio means incorporates earphones integrated with the head mounted unit.

16. Device of claim 1 wherein:
the audio signals change their intensity and frequency in correlation with intensity and frequency of the light signals created by the display means.

17. Device of claim 1 wherein:
the audio signals are chosen for their ability to induce relaxation.

18. Device of claim 1 wherein:
an additional audio signal in the form of a human voice is produced by the earphones.

19. Device of claim 1 wherein:
the control means includes a cable connection between the control unit and the head mounted unit.

20. Device of claim 1 wherein:
the control means is a wireless connection between the control unit and the head mounted unit, including a transmitting means contained in the control unit and a receiving means contained in the head mounted unit.
